(19) United States
(12) Patent Application Publication
(10) Pub. No.: US 2018/0284102 A1
(43) Pub. Date: Oct. 4, 2018

(54) DEVICES, SYSTEMS AND METHODS FOR MANIPULATING SMALL MODEL ORGANISMS

(71) Applicant: The United States Of America, as represented by the Secretary, Department Of Health&Human Services, 6011 Executive Blvd. Suite 325, MD (US)

(72) Inventors: Maria D. Jaime, Rockville, MD (US); Brian C. Oliver, Bethesda, MD (US)

(73) Assignees: The United States of America, as represented by the Secretary, Department of Health & Human Services, Rockville, MD (US); The United States of America, as represented by the Secretary, Department of Health & Human Services, Rockville, MD (US)

(21) Appl. No.: 15/526,672

(22) PCT Filed: Nov. 13, 2015
(86) PCT No.: PCT/US2015/060575

§ 371 Date: May 12, 2017

Related U.S. Application Data
(60) Provisional application No. 62/080,181, filed on Nov. 14, 2014.

(51) Int. Cl. G01N 33/50; B01L 3/00; B01L 9/00

(52) U.S. Cl. CPC G01N 33/5085; B01L 3/50255; B01L 2200/025; B01L 2300/0681; B01L 2300/0829; B01L 9/523

(57) ABSTRACT

Provided herein are a miniature devices, systems and methods for the treatment and manipulation of small model organisms to perform high-throughput screens of small molecules, chemical compounds, bioreactive agents and/or environmental conditions. Disclosed is an apparatus for housing a small model organism in array format the includes an array of chambers joined by a solid support, wherein the bottom of each chamber has a round bottom well, wherein the round bottom of the round bottom well comprises one or more holes that are: (a) of sufficiently large size to be permeable to liquid and (b) of sufficiently small size to prevent exit of the small model organism.

### CROSS-REFERENCE TO RELATED APPLICATIONS

The present application claims priority to U.S. Provisional Application No. 62/080,181, filed on Nov. 14, 2014, entitled Device and System for Manipulating Small Model Organisms. The contents of this application are incorporated by reference in its entirety.

### GOVERNMENT FUNDING

Research supporting this application was carried out by the United States of America as represented by the Secretary, Department of Health and Human Services.

### BACKGROUND OF THE INVENTION

Throughout at least the last two decades of drug research, high throughput screens and sequencing technologies have evolved considerably, due to the development of new technologies encompassing automation and large/parallel sample processing capabilities that have shortened the period of time required to obtain results and dramatically expanded the amount of data that can be obtained and processed in a fixed unit of time. However, the use of animal models remains costly due to poor throughput and excess use of test compounds, which are often limited, which has in turn limited discovery and development of novel therapeutic treatments.

The advanced genetic tools available for use in small model organisms and the evolutionary conservation of biological mechanisms and protein function across model organisms and mammals (including humans) have promoted integration of small model organisms into the drug discovery process. The utilization of model systems accelerates and facilitates performance of initial drug screening trials, allowing for more efficient discovery of putative targets and giving rise to novel therapeutic treatments that otherwise would be very costly and difficult to identify using larger animal models (i.e. rats, guinea pigs, mice, dogs, rabbits, monkeys, etc.). Nevertheless, there are some limitations in using even small model organisms on high throughput screens, due to the requirement for labor-intensive manipulation of the organisms, need for large amounts of test compounds to perform an experiment, failure to maximize on life stage treatments, growth conditions available, and the ability to be cultured and manipulated in an array format that is compatible with high-throughput sample processing platforms (e.g., 96 well plate format or higher).

There have been several attempts to address the above-recited limitations that hinder use of model organisms in high-throughput screening environments, with such attempts including the Capillary feeder (CAFE) assay, where small volumes of liquid food with treatment of interest (e.g., drugs) are provided to flies in capillary tubes inserted in the lids of narrow vials (see Ja et al., 2007). However, this kind of housing actually makes screens more labor intensive, limiting the number of samples that can be prepared, in turn limiting the number of replicates and concentrations that can be tested. Such housing also limits the range of different growth stages that can be treated to only adults.

### SUMMARY OF THE INVENTION

The present disclosure provides miniature system(s), Whole Animal Feeding Flat High-Throughput (WAFFL-HT), and method(s) that allow for reduction of small model organism manipulation, as well as reduction of drug and anesthetic use, thereby facilitating performance of automated and/or enhanced throughput manipulation and/or screening of small model organisms. The disclosed systems and methods also allow for the culture of model organisms in liquid and syrup consistency food, reduction of labor-intensive preparation for each experiment and permits organisms to be treated in situ in an array format that fits a standard microplate of an automated system/platform (e.g., containment of the small model organism within a 96 well microplate format that is directly compatible with a standard 96 well microplate). The compositions of the present disclosure thereby provide the option for automated use of small model organisms with robotic systems.

In certain aspects, the inventive disclosure specifically provides a composition for housing small model organisms (e.g., Drosophila melanogaster, Daphnia, Hyalella, C. elegans, zebrafish, etc.) in a miniature array format and methods of using such compositions, e.g., as a platform to screen for the effect(s) of administered agents (e.g., small molecules, nucleic acids, polypeptides, pathogens, etc.) and/or conditions (e.g., heat, cold, altered atmospheric conditions, vibration, magnetic fields, etc.) upon the small model organism. The arrayed housing advantageously provides a platform for performing such screening in a manner that is amenable to both automation and high-throughput screening.

One aspect of the disclosure provides an apparatus for housing a small model organism in array format that includes an array of chambers joined by a solid support, where the bottom of each chamber includes a round bottom well, where the round bottom well includes one or more holes that are: (a) of sufficiently large size to be permeable to liquid and (b) of sufficiently small size to prevent exit of the small model organism. In certain embodiments of the present disclosure the top of the array of chambers is open. Alternatively the top of the array of chambers can be covered by a removable layer, such as a mat, that is impermeable to the small model organism.

In certain embodiments, a small model organism is present in at least one chamber of the array of chambers.

Optionally, the array includes 96 chambers in an 8 row by 12 column format. However, those skilled in the art will readily appreciate that the array can include any number of chambers and be formed in various configurations/formats. For example, the array could be configured to be used with standard 6, 12, 24 or 48 well plate formats. Alternatively, the array could be configured to be non-standard or non-symmetrical and include, for example, 95 wells.

In certain embodiments, the solid support is a plastic. Optionally, the solid support is a UV cure resin, a polystyrene or polypropylene, and/or a coating of the substrate.

In certain embodiments, the small model organism is Drosophila melanogaster, Daphnia, Hyalella, C. elegans or zebrafish.

In one embodiment, the array of chambers further includes at least 3 alignment holes.

In another embodiment, the one or more holes of the round bottom well are approximately 350 microns in size.

In certain embodiments, the apparatus further includes an adapter plate also referred to herein as a transfer plate that allows for the interconnection of the array of chambers to a receiver plate, where the receiver plate includes an array of circular deep wells. Optionally, the adapter plate includes an array of square-to-round well adaptors.

Another aspect of the present disclosure provides a kit that includes an apparatus for housing a small model organism in array format, a receiver plate, and instructions for use of the kit, where the apparatus includes an array of chambers joined by a solid support, where the bottom of each chamber includes a round bottom well, where the round bottom of the round bottom well includes one or more holes that are: (a) of sufficiently large size to be permeable to liquid and (b) of sufficiently small size to prevent exit of the small model organism, and the top of the array of chambers is covered by a removable layer that is impermeable to the small model organism (silicone mats, AXYGEN®, AXYMAT™ AM-2ML-RD), and the receiver plate includes an array of circular deep wells, where the receiver plate interfaces with the apparatus directly or through use of an adapter plate.

In an additional aspect, the present disclosure provides a method for contacting a small model organism with a test compound or treatment of interest (e.g., small molecules, drugs), the method involving introducing the test compound to a standard 96 well plate and contacting the 96 well plate containing the test compound/treatment of interest with an apparatus of the present disclosure, thereby contacting a small model organism with the test compound.

Other aspects of the present disclosure are described in, or are obvious from, the following disclosure, and are within the ambit of the invention.

### BRIEF DESCRIPTION OF THE DRAWINGS

So that those having ordinary skill in the art to which the present disclosure pertains will more readily understand how to employ the systems, devices and methods of the present disclosure, embodiments thereof will be described in detail hereinbelow with reference to the drawings, wherein:

FIGS. 1A and 1B provide top and bottom views respectively of an exemplary 96 well feeder plate of the present disclosure;

FIGS. 2A-2G provide various view of an exemplary 96 well feeder plate of FIGS. 1A and 1B;

FIGS. 3A and 3B provide a top and bottom view respectively an exemplary 96 well transfer adapter of the present disclosure;

FIGS. 4A-4F provide various view of an exemplary 96 well transfer adapter of FIGS. 3A and 3B;

FIGS. 5A and 5B provide top and bottom views respectively an exemplary 96 well receiver plate of the present disclosure;

FIGS. 6A-6E provide various view of an exemplary 96 well receiver plate of FIGS. 5A and 5B;

FIGS. 7A and 7B show a complete ensemble of the exemplified 96 well miniature system of the present disclosure;

FIGS. 8A-8D provide various views of an exemplary 96 well feeder plate of the present disclosure;

FIGS. 9A and 9B provide views an exemplary 96 well transfer adapter of the present disclosure; and

FIGS. 10A-10D provide various views of an exemplary 96 well receiver plate of the present disclosure.

These and other aspects of the subject disclosure will become more readily apparent to those having ordinary skill in the art from the following detailed description of the invention taken in conjunction with the drawings.

### DETAILED DESCRIPTION OF THE PREFERRED EMBODIMENTS

Disclosed herein are detailed descriptions of specific embodiments of devices, systems, apparatus and methods for housing and manipulating model organisms. It will be understood that the disclosed embodiments are merely examples of the way in which certain aspects of the invention can be implemented and do not represent an exhaustive list of all of the ways the invention may be embodied. Indeed, it will be understood that the systems, devices and methods described herein may be embodied in various and alternative forms. Moreover, the figures are not necessarily to scale and some features may be exaggerated or minimized to show details of particular components.

Well-known components, materials or methods are not necessarily described in great detail in order to avoid obscuring the present disclosure. Any specific structural and functional details disclosed herein are not to be interpreted as limiting, but merely as a basis for the claims and as a representative basis for teaching one skilled in the art to variously employ the invention.

The present disclosure now will be described more fully, but not all embodiments of the disclosure are necessarily shown. In addition, many modifications may be made to adapt a particular situation or material to the teachings of the disclosure without departing from the essential scope thereof.

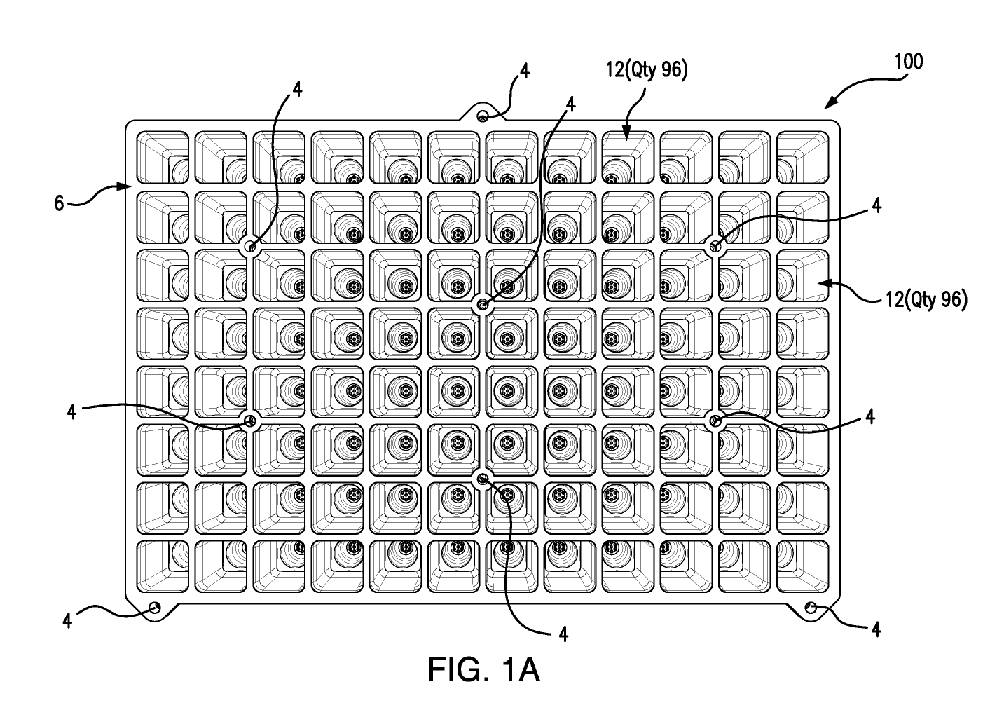

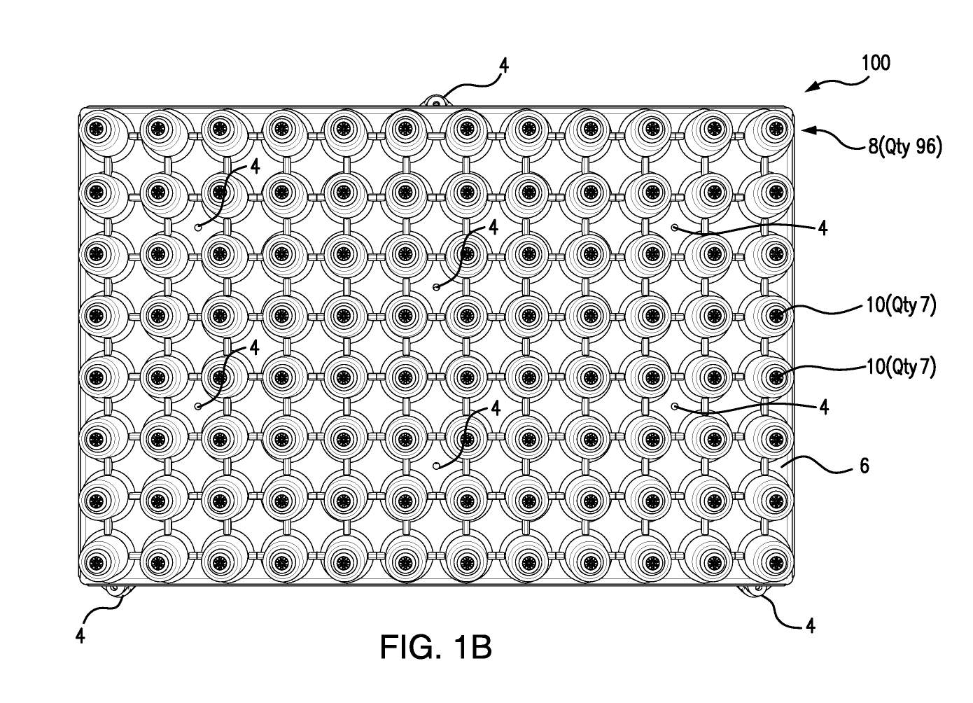

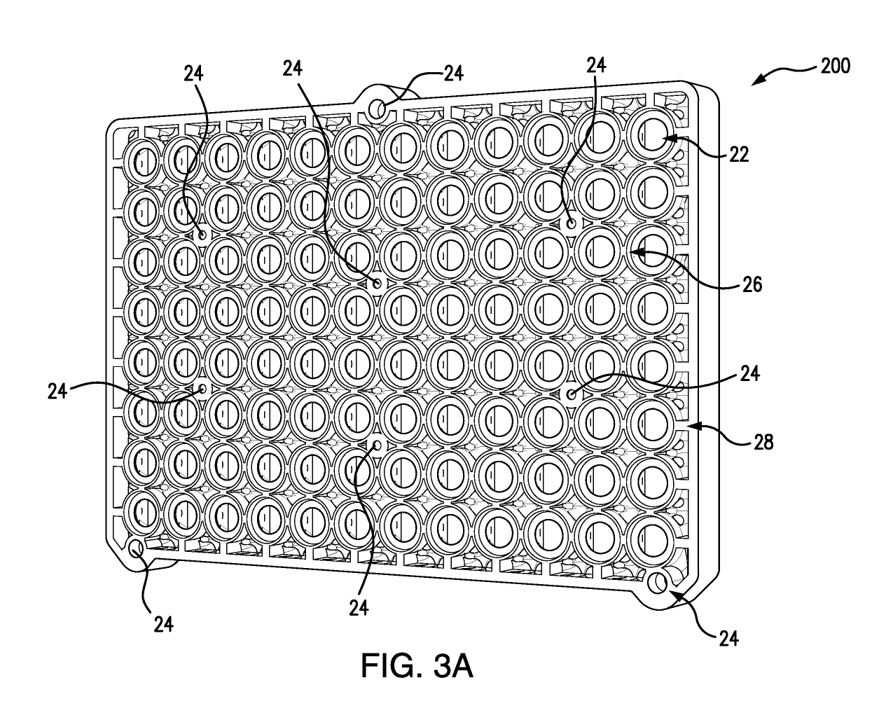

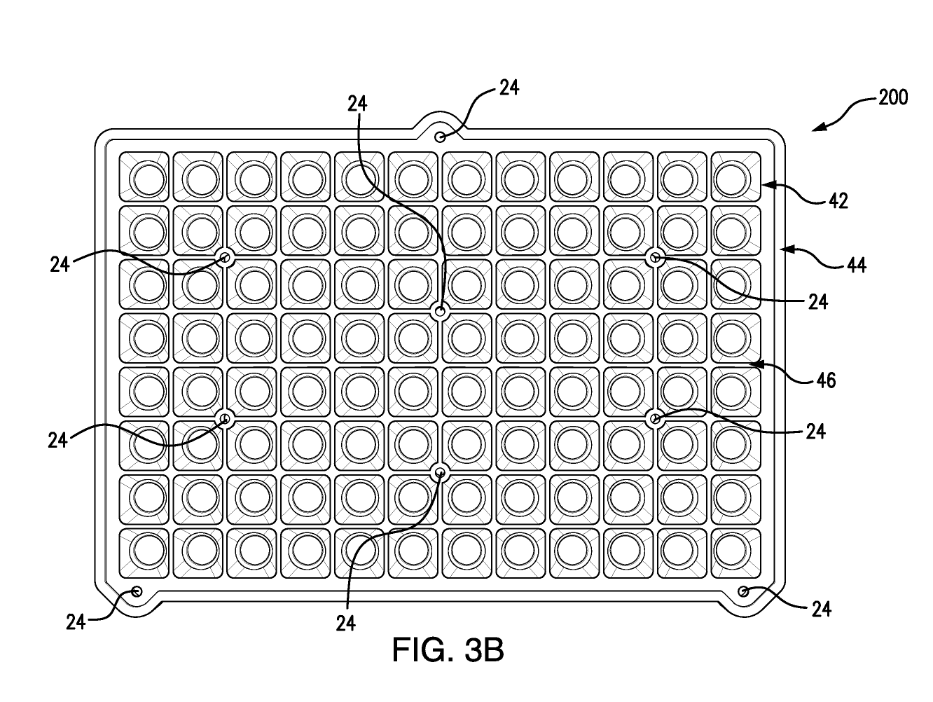

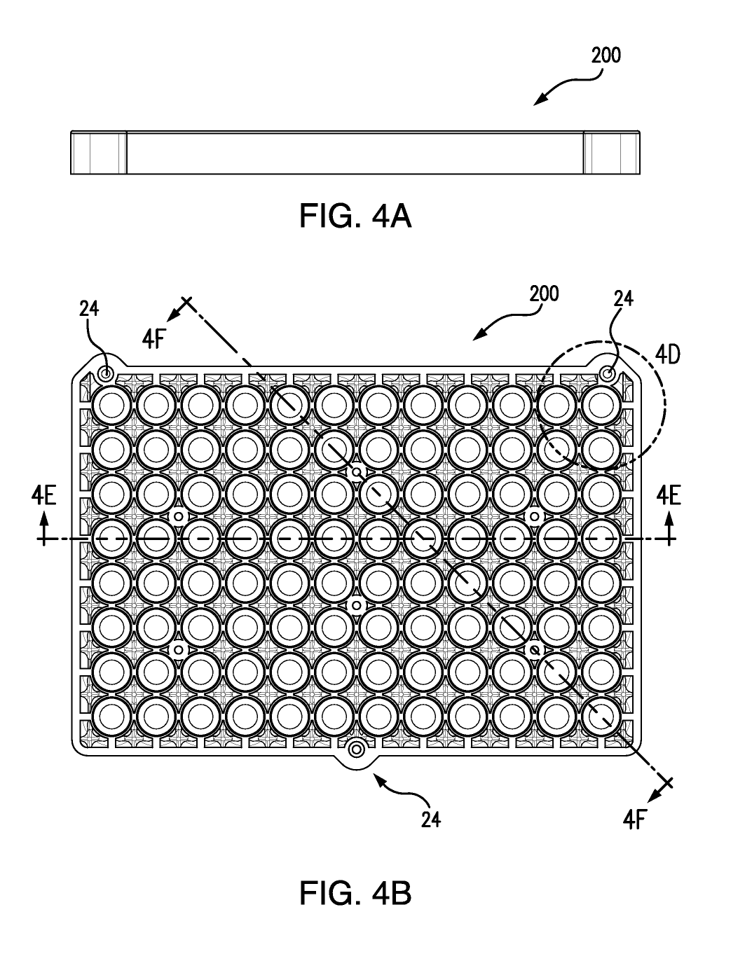

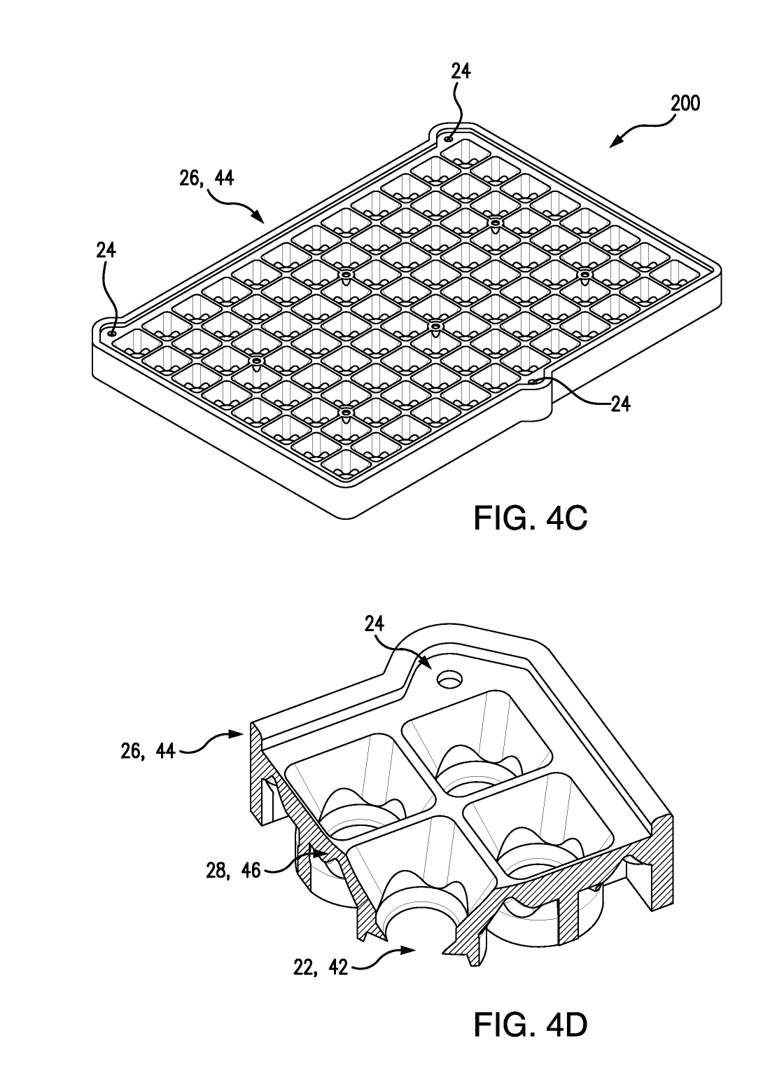

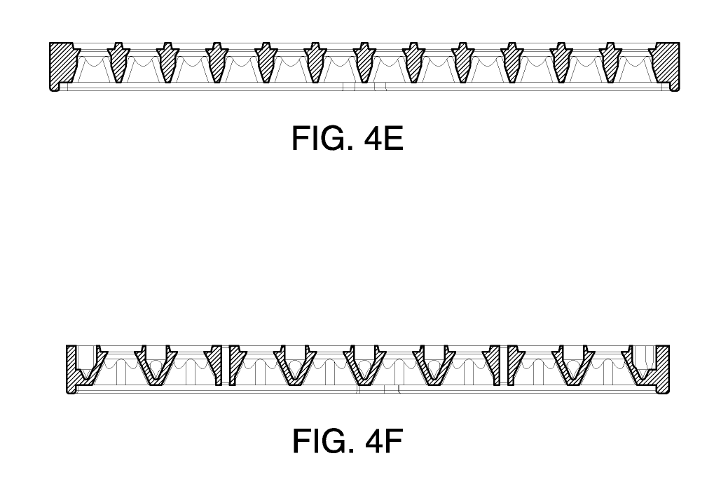

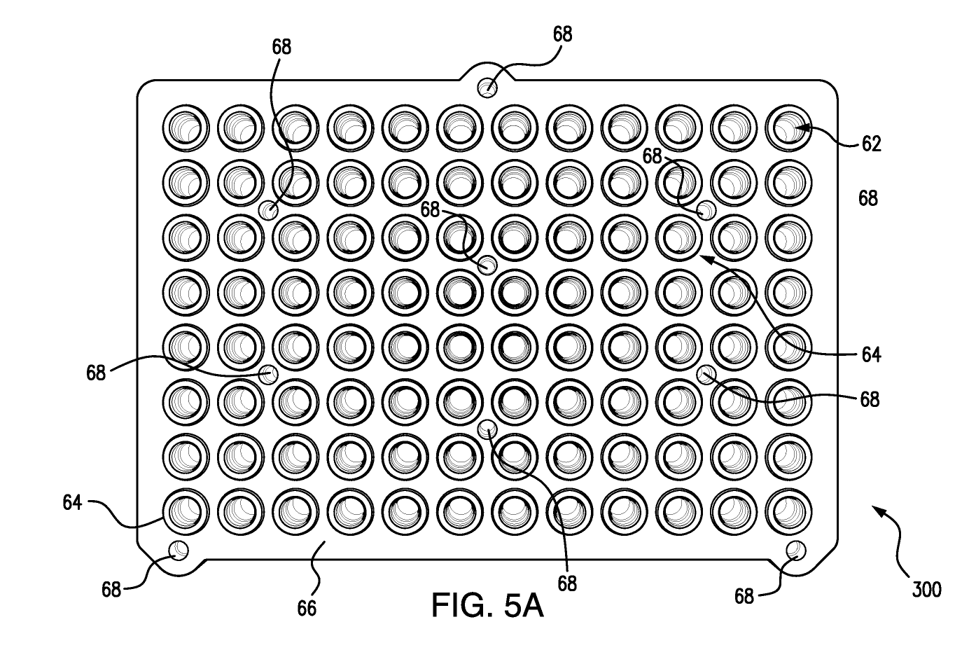

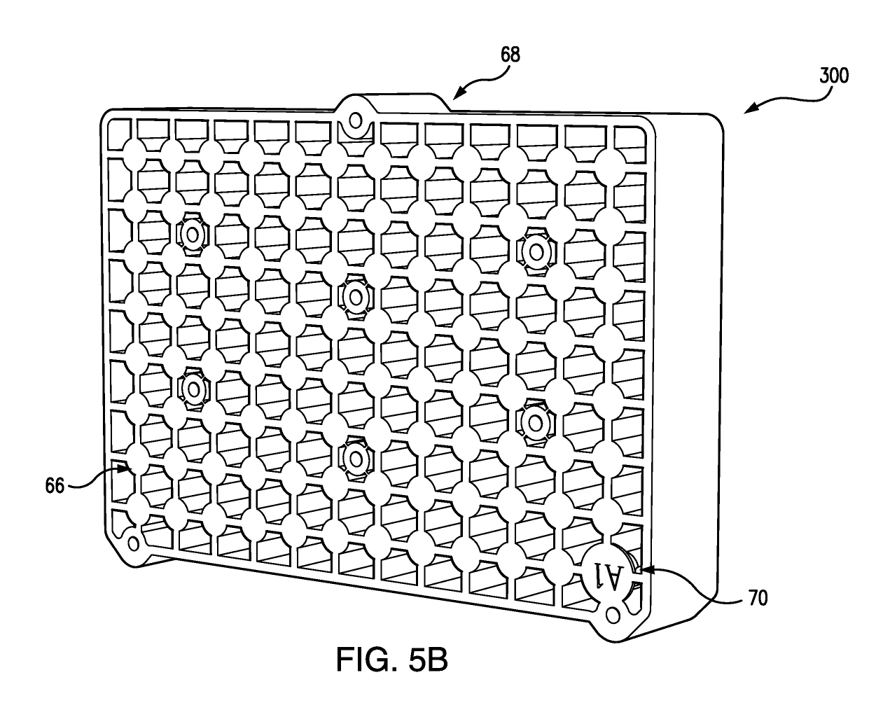

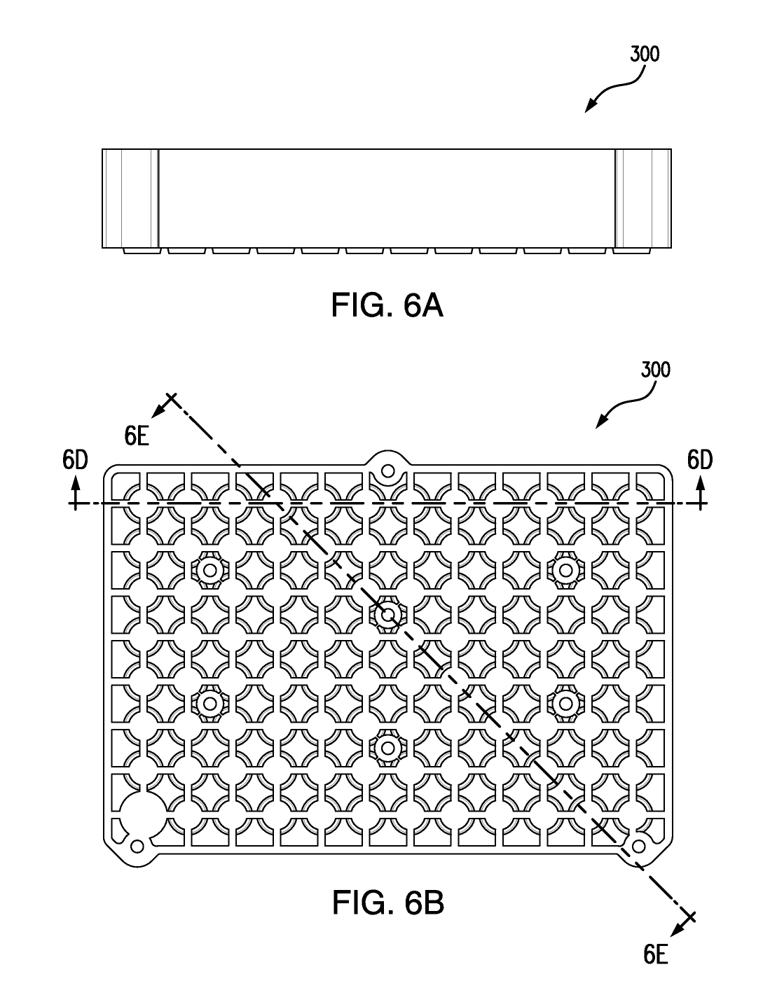

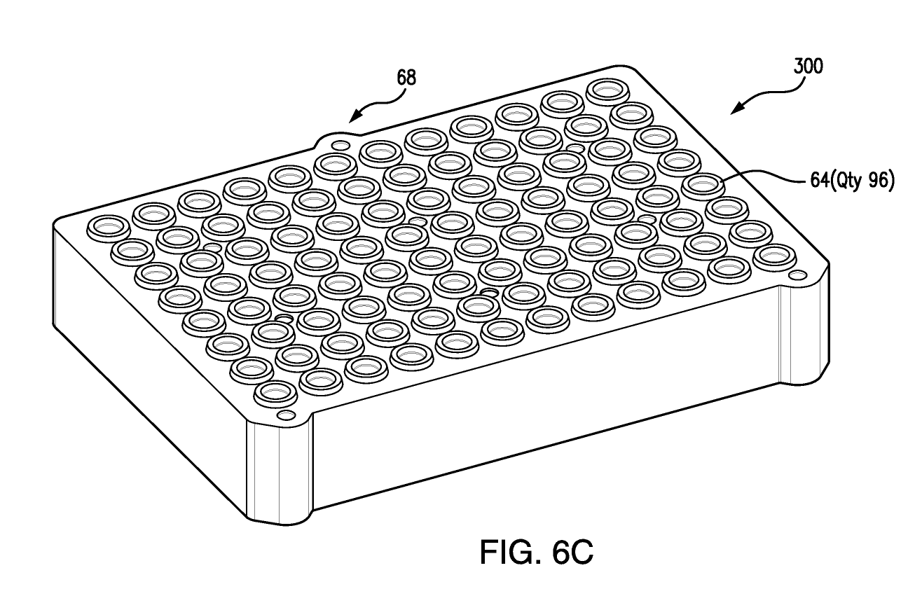

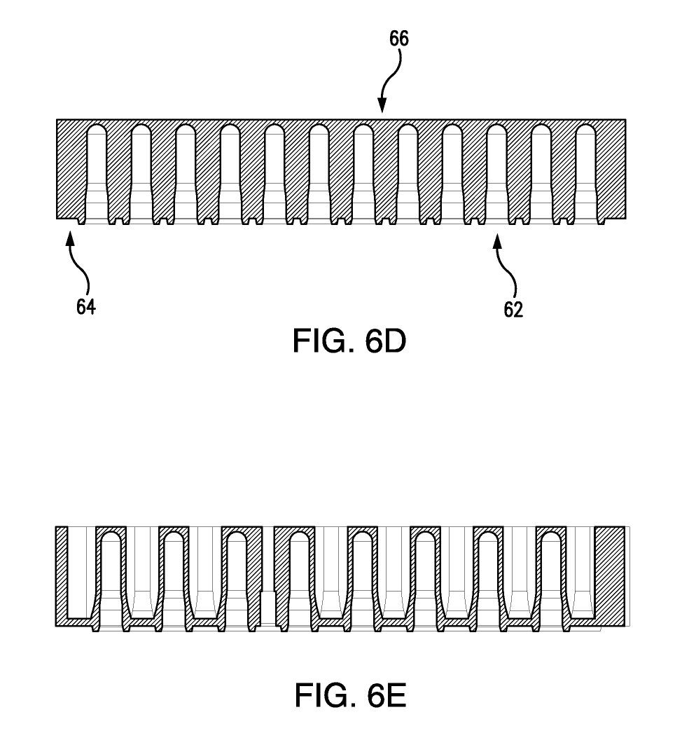

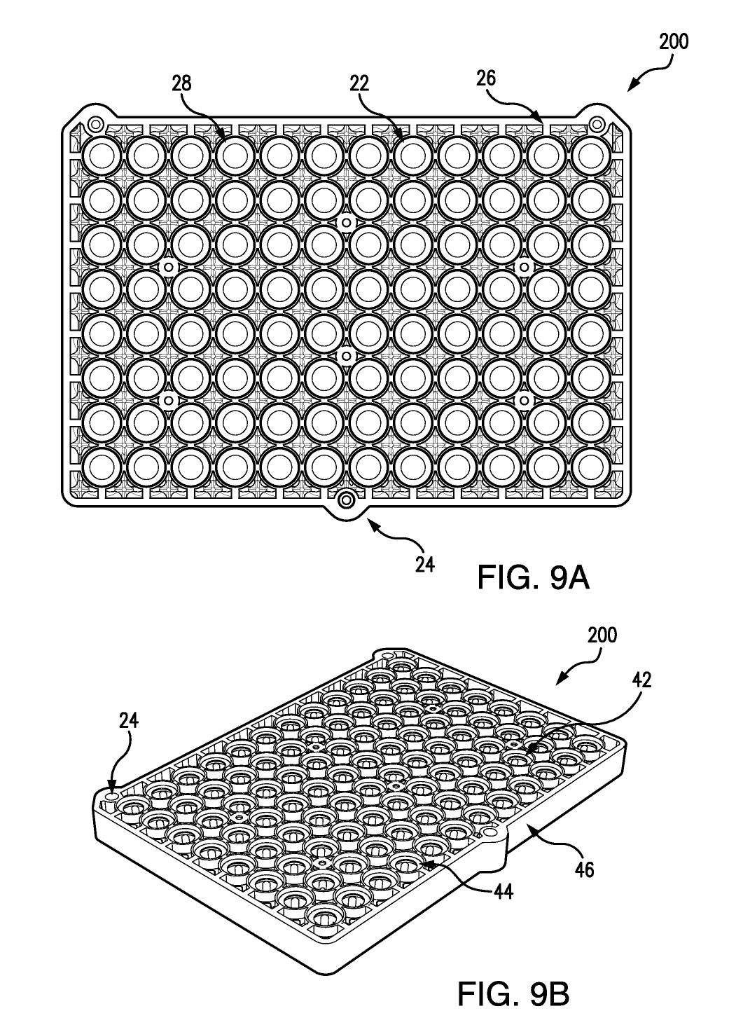

The present invention is directed, at least in part, to a miniature apparatus, device or system for housing and manipulating small model organisms in an array format which allows for use of the small model organism in automated, high throughput drug screening platforms. As will be discussed in detail below, specific embodiments of the miniature system of the present disclosure include a feeder plate having an array of chambers capable of housing the small model organism while also permitting exposure of the small model organism to food and/or test compounds presented within an array of wells (e.g., a 96 well plate comprising food and/or test compounds in a liquid state, e.g., as shown in FIGS. 1A, 1B, 2A-2G and 8A-8D) that is external to the array of chambers of the feeder plate. Embodiments of the miniature system of the present disclosure also include a transfer adapter (e.g., as shown in FIGS. 3A-3B, 4A-4F and 9A-9B) having an array of interfaces that allow the interconnection of the chambers of the feeder plate to a receiver plate, where the receiver plate has an array of circular deep wells and is optionally a location of further manipulation and/or processing of the small model organism (e.g., as shown in FIGS. 5A-5B, 6A-6E and 10A-10D).

The miniature system(s) of the present disclosure provide powerful tools that allow for screening of chemical libraries and/or other potentially bioactive agents (e.g., as potential drugs, as toxins or environmental contaminants, etc.) on small organisms that possess great diversity of available genetic tools, short life cycle and reduced cost of maintenance and culturing, as compared to murine models. Chemical screens that utilize the miniature system(s) of the present disclosure allow for discovery of bona fide targets for new therapeutic treatments, since the screens are performed in a whole organism context, rather than in cell-based assays.

The reduced cost of nurturing model organisms and reduced amount of materials required to perform high throughput screens using the miniature system(s) of the current disclosure facilitate screening of larger chemical libraries and/or environmental conditions. When such attributes are further complemented by the genetic tools available for small model organisms, where diseases can be modeled and/or reproduced via targeted genetic manipulation, limitless options for drug discovery are thereby provided. In addition, the system(s) of the invention also enable performance of experiments upon organisms of specific genotypes, allowing for evaluation and determination of optimal therapeutic treatment(s), based upon, e.g., a particular genotype and/or genomic profile of a subject, thereby allowing for practice of personalized medicine.

The miniature system(s) of the present disclosure fill a gap between cell-based high throughput screens of chemical libraries, which have become highly automated in recent years, and validation of putative therapeutic treatments on murine or higher animals, which remains a relatively labor intensive and costly process.

### Definitions

Unless defined otherwise, all technical and scientific terms used herein have the meaning commonly understood by a person skilled in the art to which the present disclosure appertains. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

About: As used herein, the term “about” means+/−10% of the recited value. Use of “about” is contemplated in reference to all ranges and values recited herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The present disclosure is primarily relevant to the fields of automated drug screen systems and drug screen assay techniques, as the disclosed devices specifically provides a system that enables the implementation of high throughput screens of chemical libraries and other environmental conditions upon a small model organism housed within an array.

The miniature system enables the manipulation of small organisms in an array format that is compatible with automated/robotic platforms (e.g., 96 well plate format, though other array formats are also contemplated, e.g., 384 well or other). While capabilities of the miniature system of the invention have initially been characterized using the model organism Drosophila melanogaster, modification of the current system to suit other small model organisms at different growth stages, such as Daphnia, Hyalella, C. elegans and zebrafish, is also contemplated and can be performed readily. In exemplified aspects, the miniature system of the invention permits the treatment and manipulation of adult flies without the need for use of CO₂ or anesthetics that significantly alter behavior and transcriptional profiles of flies.

The system also enables the culture of organisms using very small volumes of liquid medium (e.g., less than 20 microliters), reducing the amount of drug or other treatments needed for screening, as well as limiting the waste of treatment compounds. The system facilitates the transfer of the organisms from one condition to another in seconds by simply placing the feeder plate on a different microplate (e.g., 96 well microplate) with the condition of interest. The option of being able to grow adult flies in liquid medium and manipulate them in an array (e.g., 96 well) format overcomes the two major limitations for use of D. melanogaster adults in high-throughput screens. Similar limitations also affect other model organisms, providing broad applicability of the current system to researchers in many areas.

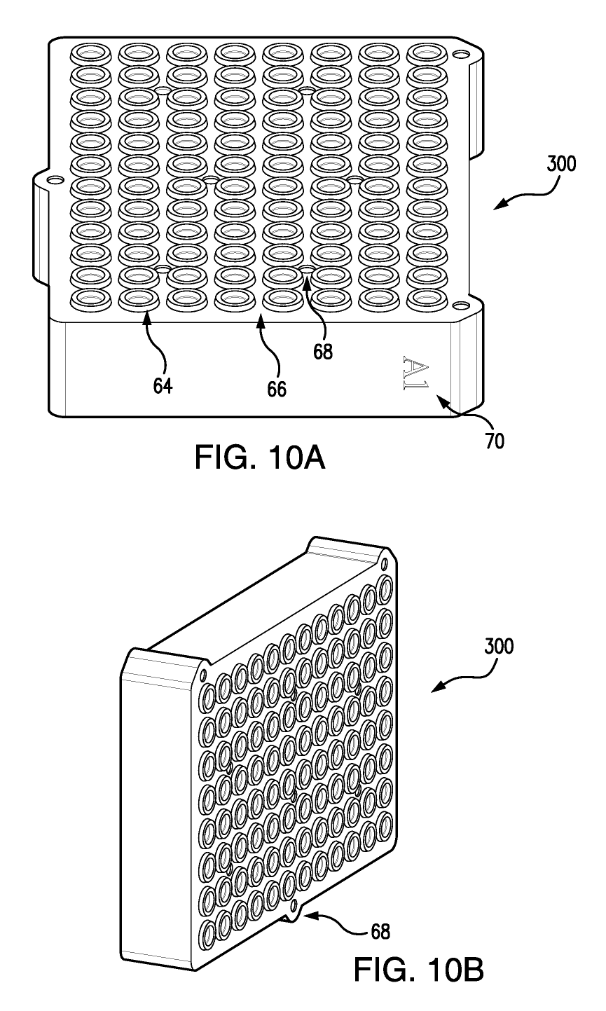

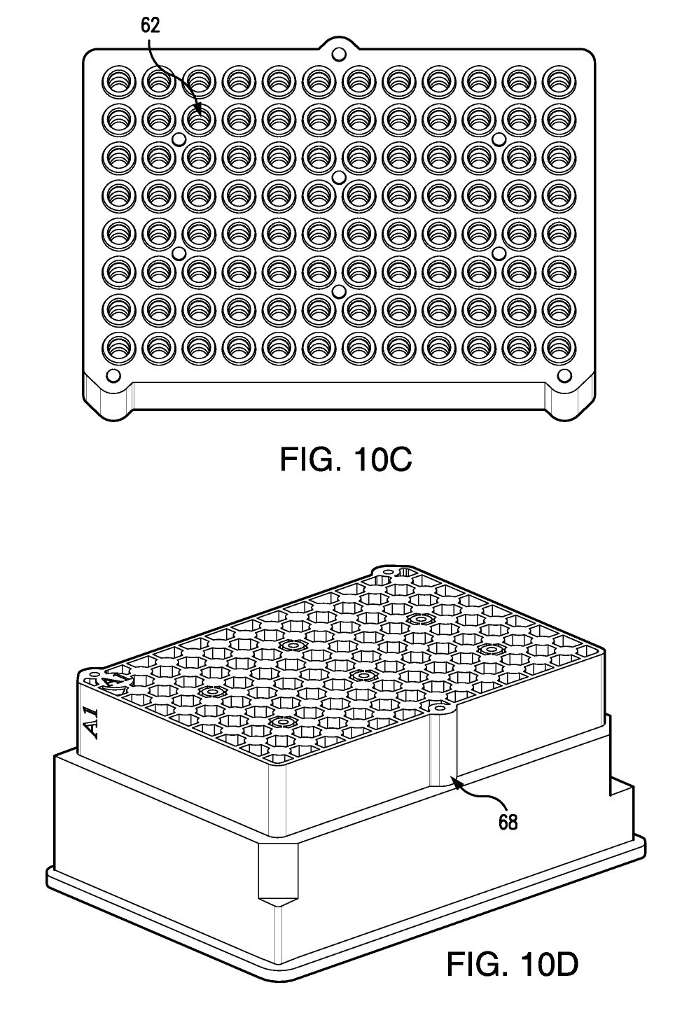

The currently described system possesses the attribute of reducing the manipulation of small model organisms, since they live their adulthood and/or complete life cycle in the miniature system, where they can be treated in situ. Manipulation is further reduced at the end of the experiment via use of the transfer adaptor and receiver plate, which facilitate immediate and quick transfer of the organisms to a standard deep well plate where RNA/DNA/metabolite extraction protocols can be performed (see, e.g., FIG. 10D). Minimization of organism manipulation is important because it reduces the possibility of alteration of transcriptional profiles and/or infliction of stress in the model organisms, while also reducing the amount of labor required to perform experiments upon the model organisms.

The miniature systems of the invention therefore enable manipulation and treatment of small model organisms in an efficient manner that reduces time and consumables used for high-throughput screens.

Further attributes and certain optional modifications of the miniature system(s) of the present disclosure are described in greater detail below.

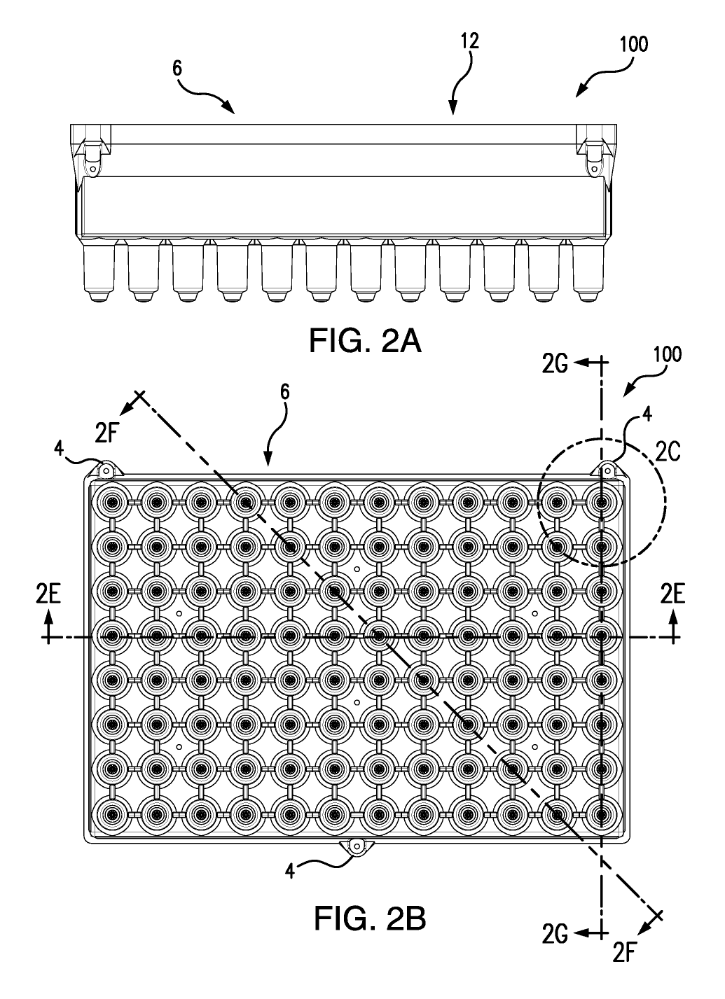

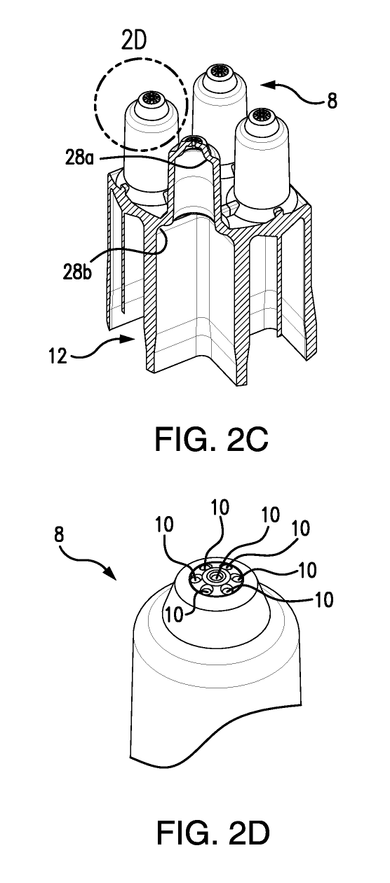

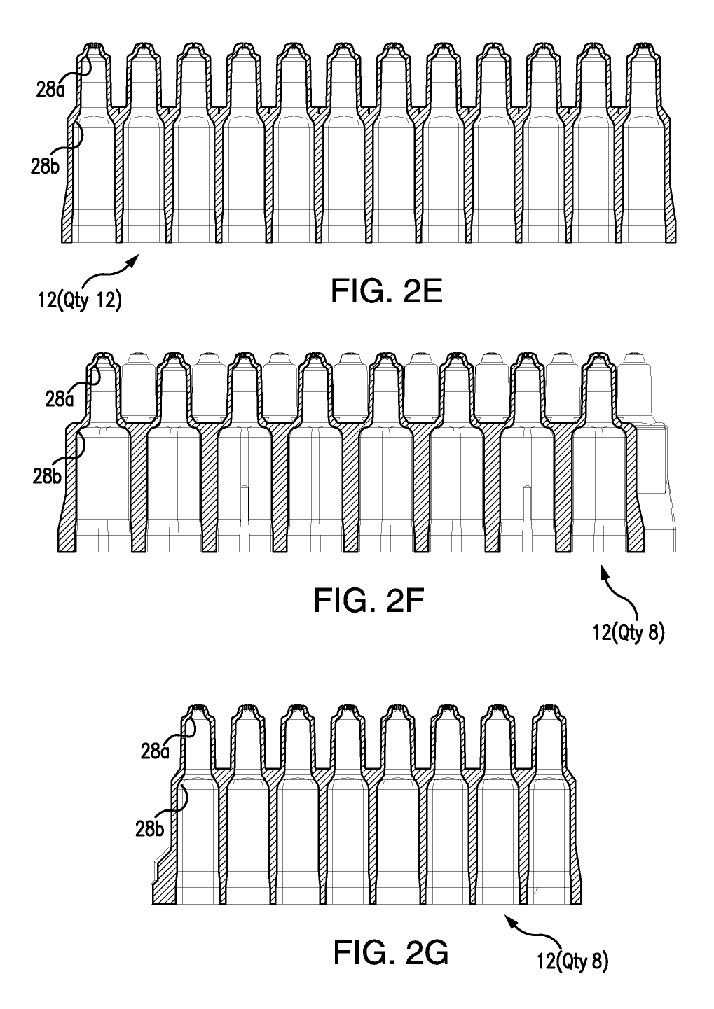

Referring now to FIGS. 1A through 2G which illustrate several views of a feeder plate which has been constructed in accordance with an embodiment of the present invention and designate as reference numeral (100). As shown in these figures, feeder plate (100) includes an array of chambers (12) or wells that terminate in a round well bottom (8). Each round well bottom (8) has seven holes (10) of 350 microns in diameter. It is explicitly contemplated that any number of holes between one and twenty or more can readily be employed in the feeder plates of the present disclosure, with pore sizes optionally in any size range from as small as 1 micron or less to approximately 0.5 mm or more (provided that the hole size is not sufficiently large to allow egress of the small model organism from the feeder plate round bottom holes). Thus, exemplary hole sizes include about 1 micron, 2 microns, 5 microns, 10 microns, 20 microns, 30 microns, 40 microns, 50 microns, 100 microns, 150 microns, 200 microns, 250 microns, 300 microns, 350 microns, 400 microns, 450 microns and 500 microns or more.

As shown in FIG. 1A, in certain embodiments, feeder plate (100) includes nine orifices (4) where alloy steel dowels can be fitted and then sealed with clear silicone that allows the feeder plate (100) to fit with complementary pieces of the miniature system (transfer adapter and receiver plate). Such orifices/holes (4) can also be used for alignment of the plate(s) to an automated/robotic platform more generally (e.g., robotic platform for automated manipulation of the feeder plate, or of other plates of the miniature system that possess holes that can be used for plate alignment). While nine orifices (4) are used in certain plate examples of the present disclosure, it is explicitly contemplated that any number of holes between one and twenty or more (1, 2, 3, 4, 5, 6, 7, 8, 9, 10, 11, 12, 13, 14, 15, 16, 17, 18, 19, 20 or more) can be employed for such plate alignment purpose(s). In certain embodiments, at least three such alignment holes are present in a feeder plate and/or transfer adapter and/or receiver plate of a miniature system of the present disclosure. The alignment orifices/holes (4) also help to avoid an errant rotation of the entire plate, since they have a unique alignment to ensure the correct orientation of the plates.

The feeder plate (100) and the remaining elements of miniature system (500) of the present disclosure were designed for use with small model organisms. It is contemplated that initial introduction of small model organisms to a feeder plate of the current disclosure can be achieved by one or more of the following means:

(1) Placement of an egg and/or larval and/or pupa form of a small model organism into each well (12) of a feeder plate (100), with food provided via penetration of the round well bottoms (8) of the feeder plate (100) upon contact with an arrayed base plate (e.g., standard 96 well plate) containing food.

In certain embodiments that are most directly relevant to housing flies in a feeder plate of the present disclosure, ten to eleven days post-egg laying, brown pupae are removed with a wet fine brush from Drosophila culture vials and are optionally sorted by sex. Males can be differentiated from females based upon the presence of sex combs. Adult flies are fed with chemical defined food (see Lee and Micchelli, 2013) with the minor modification of not adding agarose, thereby providing food in liquid form. Flies can be fed daily with 10 ul of chemical defined liquid fly food (CDF) 14 hours after pupae were placed in the well and adults have eclosed. After 2 days of regular feeding, flies can be starved for up to 12 hours, allowed to drink only PBS pH 7.4 during such period, then flies can be fed with CDF with dye (e.g., 0.5 mg/ml of Sulforhodamine B as well as test compound(s) of interest. The use of Sulforhodamine B allows one to identify feed from un-feed flies as well as the leftover volume of food with treatment after feeding the flies. In the specific case of flies, the holes (10) in the round well (8) are large enough to allow adult flies to have access to the food but small enough to avoid that the flies get their wings wet or drown. As shown in FIGS. 2E-2G, in certain embodiments, the feeder plates of the present disclosure effectively include inner ledges (28a, 28b) upon which a housed fly can perch—removed from the liquid food below—while extending its proboscis into the liquid food to eat. It is contemplated that different growth stages (such as embryo, larvae) can be accommodated in a feeder plate of the disclosure, optionally by modifying the style of the round wells of the feeder plate to allow the small organism to have access to gelled food.

In comparison to a regular narrow vial assay which requires approximately 1 ml of food in the vial for a single treatment, the feeder plate of the present disclosure only requires 1 ml of food for 96 treatments.

While liquid food is used in the above-described embodiments, it is also contemplated that for experiments performed upon embryos or larvae, solid food can be used via addition of agarose or agar to the chemical defined food used to feed adult animals, thereby preventing drowning of embryo and/or larval forms of small model organism.

(2) The miniature system also offers the possibility of placing individual adult flies in separate chambers or wells (12) of feeder plate (100). Here, it is contemplated that CO₂ or other anesthetic might be used to introduce flies to wells of the feeder plate, but that the flies might then recover for a sufficient period of time (e.g., 12 h to 24 h) post-anesthetic before contacting with test compounds or other conditions in performance of screening. The structure and characteristics of the miniature system of the present disclosure also provide the possibility of administering a treatment by inhalation, since gases can also penetrate from the holes in the bottom of the well to treat the small organisms.

In certain embodiments most relevant to Drosophila, once flies have been treated, harvest of the flies can be performed in the absence of CO₂ or other anesthetic by transferred a feeder plate housing flies to −20° C. or −80° C. for at least 15 min to immobilize the flies, then the silicone mat which is used to cover the wells of the feeder plate (100) can be removed, and the transfer adapter (200) and receiver plates (300) (discussed in detail later) can be attached to the feeder plate (100).

Flies can then be transferred by centrifugation for 10-20 seconds at less than 1000 rpm. Optionally, once familiar with the manipulation of the miniature system, the harvest of flies can also be performed by softly tapping the feeder plate (100) to bring the flies to the bottom of the well(s) (12), quickly removing the silicone mat, and then attaching the transfer adapter (200) and receiver plate (300), prior to centrifugation or further tapping. To ensure that the flies don't fly out of the receiver plate (300), a 5 ul droplet of lysis buffer or ultrapure water can optionally be placed in the well so that flies that enter the wells get wet and are not able to escape. As shown in the figures and discussed below, the reduced narrow wells (62) of the receiver plate (300) are intended to restrict the movement of the flies (or other small model organism), also reducing the probability of having flies (or other small model organism) escape.

In certain embodiments, once small model organisms are in the receiver plate (300), the receiver plate (300) can be attached to a standard 96 deep well plate (10D), then flies can be transferred to this plate by centrifugation, and the samples can optionally then be frozen, or used directly to proceed with RNA/DNA/metabolite isolation.

It is contemplated that empirical tests can be used to define experimental conditions that are useful for the optimal performance of the miniature system(s) of the present disclosure, such as fly density per well, quantification of drug intake, volume of food and/or type of food. Upon defining such baseline conditions, feeding of different diets (composition and quantity), as well as administration of existing FDA-approved pharmaceuticals, lead compounds in development, and expanded chemical libraries can be performed upon small model organisms manipulated within the miniature system(s) of the present disclosure. It is explicitly contemplated that such administration can be directed towards understanding of lipid droplet function, as well as toward administration of environmental toxins, as approaches to validate the functionality and usability of the system of the invention.

In exemplified formats, Stereolithography (SLA) 3D printing can be used to produce the arrays and components of the present disclosure. It is contemplated that the currently exemplified system (or any system of the present disclosure) could be made in any SLA 3D printer capable of printing pieces with high resolution (at least layer (Z) resolution of 0.05 mm; In plane (X-Y) resolution of 0.08 mm Plastic injection molding also can be used to manufacture plates, and some of the features of the currently exemplified aspects of the invention, such as the 350 micron orifices in the round wells of the below examples, could be made by laser cutting. It is further contemplated that the miniature system pieces of the disclosure could be made of different resins or materials, such as UV cure resin, polystyrene or polypropylene, coating of the substrate, and that different degrees of opaqueness and colors for the resins and/or materials can be used, depending upon the purpose of the experiments and/or conditions where the miniature system is going to be used.

The systems described herein have several if not all of the following distinctive attributes:
Manipulation of flies or other small organism without using anesthetics,
Use of reduced volumes of small molecules, chemical compounds and/or other potentially bioactive agents to perform experiments,
Reduced waste of precious small molecules, chemical compounds and/or other potentially bioactive agents,
Allows the culture of adult flies in liquid food,
Considerably reduces the time of preparation and manipulation of the organism during experimentation,
Allows the treatment of an individual small model organism in a standard 96 well microplate format, using commercially available 96 well microplates.
Provides a means of automating test protocols using robotic systems to perform experiments with a model organism that it was heretofore not feasible.

The following publications are exemplary in defining methods currently used for the evaluation of small molecules, chemical compounds and environmental conditions with certain small model organisms and the contents of which are herein incorporated by reference. The status of the use of small model organisms for the discovery of new therapeutic treatments and the limitations faced in contemplating use of these organisms is also disclosed. Such limitations are overcome by the miniature system(s) of the current disclosure.
Carroll, P. M., et al. (2003). “Model systems in drug discovery: chemical genetics meets genomics.” Pharmacol Ther 99(2): 183-220
Chamilos, G., et al. (2011). “Drosophila melanogaster as a model host for the study of microbial pathogenicity and the discovery of novel antimicrobial compounds.” Curr Pharm Des 17(13): 1246-1253
Desalermos, A., et al. (2011). “Using Caenorhabditis elegans for antimicrobial drug discovery.” Expert Opinion on Drug Discovery 6(6): 645-652. (Perwitasari, Bakre et al. 2013)
Giacomotto, J. and L. Segalat (2010). “High-throughput screening and small animal models, where (Lionakis 2011) are we?” Br J Pharmacol 160(2): 204-216.
Gladstone, M. and T. T. Su (2011). “Chemical genetics and drug screening in Drosophila cancer models.” J Genet Genomics 38(10): 497-504.
Gonzalez, C. (2013). “Drosophila melanogaster: a model and a tool to investigate malignancy and identify new therapeutics.” Nat Rev Cancer 13(3): 172-183
Ja, W. W., et al. (2007). “Prandiology of Drosophila and the CAFE assay.” Proc Natl Acad Sci USA 104(20): 8253-8256.
Lionakis, M. S. (2011). “Drosophila and Galleria insect model hosts: new tools for the study of fungal virulence, pharmacology and immunology.” Virulence 2(6): 521-527
Perrimon, N., et al. (2007). “Drug-target identification in Drosophila cells: combining high-throughout RNAi and small-molecule screens.” Drug Discov Today 12(1-2): 28-33
Perwitasari, 0., et al. (2013). “siRNA genome screening approaches to therapeutic drug repositioning.” Pharmaceuticals 6(2): 124-160.
Pukkila-Worley, R., et al. (2009). “Antifungal drug discovery through the study of invertebrate model hosts.” Curr Med Chem 16(13): 1588-1595.
Seabra, R. and N. Bhogal (2009). “Hospital infections, animal models and alternatives.” European Journal of Clinical Microbiology and Infectious Diseases 28(6): 561-568.
Tickoo, S. and S. Russell (2002). “Drosophila melanogaster as a model system for drug discovery and pathway screening.” Curr Opin Pharmacol 2(5): 555-560.
Willoughby, L. F., et al. (2013). “An in vivo large-scale chemical screening platform using Drosophila for anti-cancer drug discovery.” Dis Model Mech 6(2): 521-529.
WO 2005/069872 A2, Title: High throughput pharmaceutical screening using Drosophila.
US 2002/0026648 A1, Title: Function-based small molecular weight compound screening system in drosophila melanogaster.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000). Drosophila: A Laboratory Handbook (Michael Ashburner, Kent Golic and R. Scott Hawley), (2^{nd} Ed., Cold Spring Harbor Laboratory Press, 2004). Exemplary Drosophila propagation and manipulation references include: Ja et al. (Proc Natl Acad Sci USA 104(20): 8253-8256) and Lee and Micchelli (PLoS One 8(7): e67308).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The following examples are put forth so as to provide those of ordinary skill in the art with a complete disclosure and description of how to make and use the assay, screening, and therapeutic methods of the present disclosure, and are not intended to limit the scope of what the inventors regard as their invention.

### EXAMPLES

The present disclosure is described by reference to the following Examples, which are offered by way of illustration and are not intended to limit the invention in any manner. Standard techniques well known in the art or the techniques specifically described below were utilized.

### Example 1. Design and Use of a 96 Well Feeder Plate for Housing Small Model Organisms

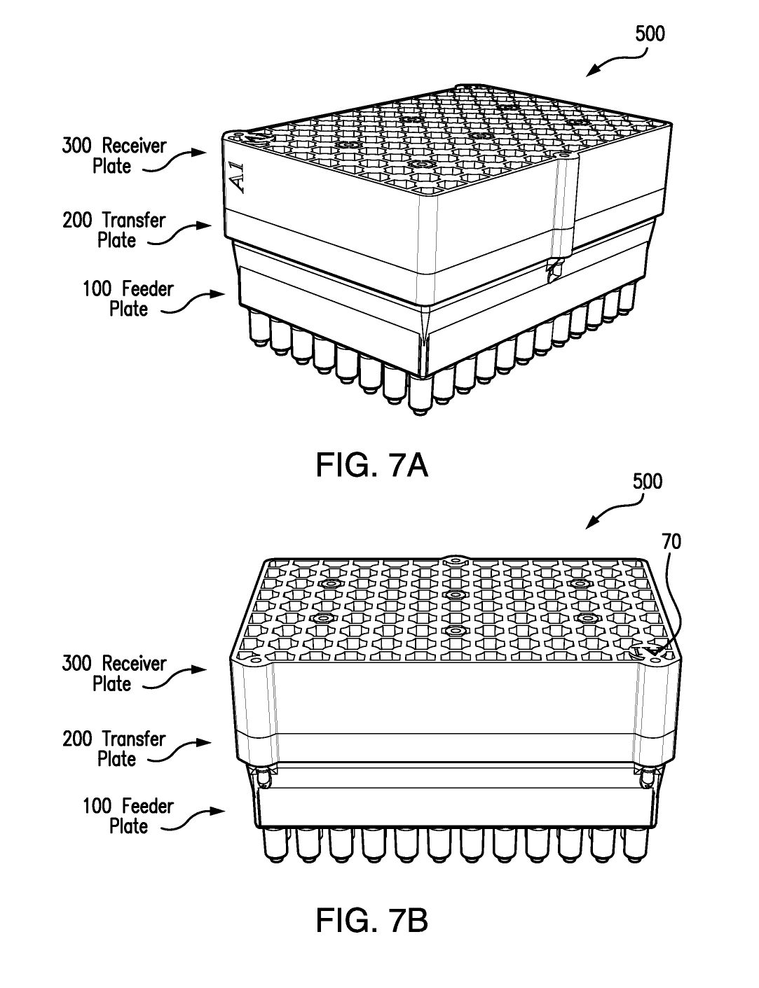

A 96 well miniature system for housing, manipulating and treating small model organisms which has been designated as reference numeral (500) (FIGS. 7A-7B) was designed and constructed, having the following core components: A) a feeder plate (100) (FIGS. 1A-2G and 8A), B) a transfer adapter (200) (FIGS. 3A-4F and 9A-B) and C) a receiver plate (300) (FIGS. 5A-6E and 10C-D).

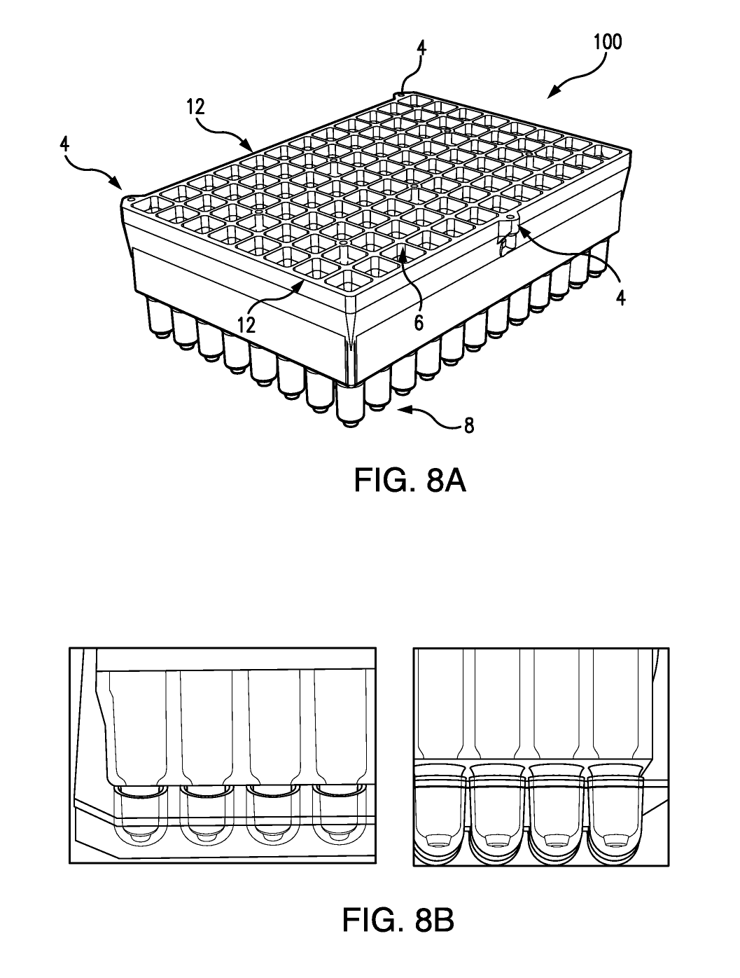

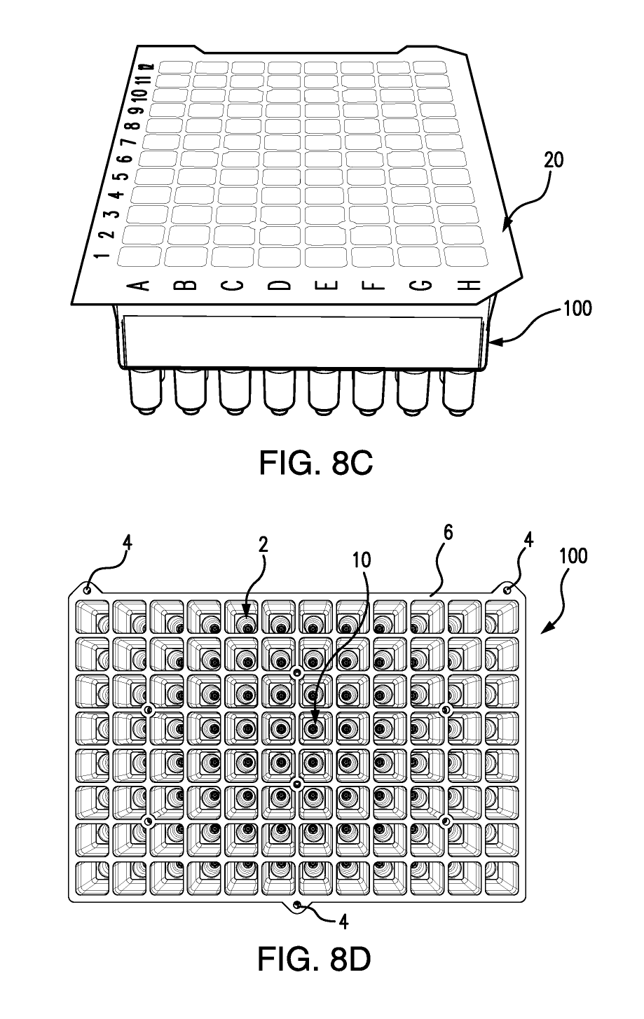

The feeder plate (100) is a 96 well plate having square sides (112) with round-bottomed wells (12) that fit in a standard 96 well microplate having a round or flat bottom (FIG. 8B, Polystyrene or polypropylene). However, as noted previously, those skilled in the art will readily appreciate that the feeder plate (100) array can include any number of chambers and be formed in various configurations/formats. For example, the array could be configured to be used with standard 6, 12, 24 or 48 well plate formats. Alternatively, the array could be configured to be non-standard or non-symmetrical and include, for example, 95 wells.

Individual wells (12) of the feeder plate (100) are joined together with a solid support (6, e.g., comprising plastic, resin, silicone, polystyrene, polypropylene, etc.) to form an array. As best shown in FIG. 2D, each round well bottom (8) of the currently exemplified feeder plate has 7 holes (10) of 350 microns (sizes can vary depending on the developmental stage and/or the particular model organism housed) that permit liquid, syrup consistency and/or solid food, and/or a treatment (e.g., a test compound) present in the standard 96 well plate to be accessible to the organism.

The top part of each well in the feeder plate (100) includes a deep square shaped section where a fly or any other small model organism housed in an individual array chamber has sufficient space to move, such that it can have a normal life cycle minimizing stress (FIG. 2C). The exemplified feeder plate (100) also includes 9 orifices (4) where alloy steel dowels can be fitted, and the feeder plate (100, FIG. 1A) can be sealed with clear silicone that allows it to fit with the complementary pieces (transfer adapter and receiver plate, e.g., as described below). In the exemplified miniature system, no magnets were used in the feeder plate, to avoid any alteration in transcriptional profiles of housed model organisms due the presence of a magnetic field. The feeder plate (100) was designed in such a way that it could be sealed using commercially available silicone mats (FIG. 8C, AXYGEN®, AXYMAT™ AM-2ML-RD). In the feeder plates of the present disclosure, the small model organisms could be housed, fed and treated in situ to perform high throughput assays.

It is contemplated (and has been incorporated into certain feeder plate designs) that depending upon the equipment, materials used to make the feeder plate and other designs that might be desirable for a miniature system of the present disclosure; sizes of the various components can be adjusted. For example, the length of the plate can optionally range from 110-128 mm. The length from the center of well one to the center of well twelve can optionally be from 97-100 mm. The width from the center of A1 well to the center of H1 well can optionally be 62 to 66 mm. The width between the edge holes for the dowels can optionally be between 73-86 mm. The height of the wells can also be adjusted: depending upon the experiment and the equipment, well height can optionally range from 30 to 50 mm from the top of the square well to the tip of the round well. From the top of the square well to the round well before the tip reduction can optionally be from 32-50 mm. The round well length can optionally range from 10-15 mm.

It should be appreciated that the number of wells in the feeder plate could be modified, for example, to 48 or 24 to be able to house larger model organisms or larger populations of organisms. However the dimension of the feeder plate, specifically the bottom part of the feeder plate, can still be configured to fit in the regular 96 well plate. Such as with 48 wells, each well will have two wells of the 96 plate and 24 will have 4 round wells. This option also provides the possibility to perform behavioral assays providing different treatments in each well and see which treatment (e.g. color, food, odor, etc) is selected.

### Example 2. Design and Use of Transfer Adapter and Receiver Plate Components Compatible with a 96 Well Feeder Plate

The other two components of the miniature system (500) were designed and developed to facilitate the manipulation of the small model organisms housed in the 96 well feeder plate (100), particularly for further processing of such organisms after experimentation. The transfer adapter (200) and receiver plate (300) components of the present example are complementary pieces to the feeder plate (100) that interconnect by alloy steel dowels and magnets.

Transfer adapter (200) was designed and constructed as a 96 well interface (FIGS. 3A-4F and 9A-9B) that allows the interconnection of the feeder plate (100) to the receiver plate (300). This piece is a square-to-round well (22, 42) adaptor that allows the transfer of organisms from the feeder plate (100) (upper square well section) to the receiver plate (300) (round well section). The transfer adapter (200) of the current example has 9 orifices (24) for magnets or dowels that allow it to fit with the feeder plate (100) and receiver plate (300) (optionally, with either plate in isolation, or with both feeder plate and receiver plate together). The alignment of the orifices is asymmetric, thereby ensuring that the pieces assemble only in the correct orientation. The transfer adapter (200) also has a tongue (26) (front face) and a groove (44) rear face) formed around the periphery of the wells to interconnect with corresponding features formed in the feeder plate (100) and receiver plate (300). Individual square-to-round well adaptors of the transfer adapter (200) are joined together with a solid support (28, 46, e.g., comprising plastic, resin, silicone, etc.) to form an array.

It is contemplated (and has been incorporated into certain transfer adapter designs) that depending upon the equipment, materials used to make the transfer adapter (200) and other designs that might be desirable for a miniature system of the present disclosure; sizes of the various components can be adjusted. For example, the length of the transfer adapter can optionally range from 113-127 mm. The length from the center of well one to the center of well twelve can optionally range from 97-100 mm. The width from the center of A1 well to the center of H1 well can optionally range from 62 to 66 mm. The height of the adapter can optionally range from 8-10 mm. The height of the lip on the round side of the adapter can optionally range from 1.3-1.8 mm. The rest of measurements follow the same sizes as the feeder plate.

As shown in FIGS. 5A-6E and 10, receiver plate (300) was designed and constructed as a 96 well feature having circular narrow deep wells (62), where flies can be relocated by centrifugation or light tapping from the feeder plate (100) to then be transferred to a 1.1 or 2 ml deep well plate (Axygen P-OW-11-C-S or P-DW-20-C-S) for RNA/DNA/metabolite isolation. The receiver plate (300) has tongue (64) around the perimeter of each well (62) that fits into a corresponding groove (44) of the transfer adapter (200) to ensure the transfer of the flies to the corresponding well, as well as into the transfer adapter. The receiver plate (300) of the current example has 9 orifices (68) for magnets or dowels that allow it to fit with the holes (24) formed in the transfer adapter (200). Individual circular narrow deep wells (62) of the receiver plate are joined together with a solid support (66, e.g., comprising plastic, resin, silicone, polystyrene, polypropylene, etc.) to form an array. As shown in FIG. 10A, the receiver plate (300) has a label in the A1 (70) well to aid orientation of the plate, thereby avoiding rotation of the experimental samples.

It is contemplated (and has been incorporated into certain receiver plate designs) that depending upon the equipment, materials used to make the receiver plate (300) and other designs that might be desirable for a miniature system of the invention, sizes of the various components can be adjusted. For example, the length of the receiver plate can optionally range from 113-127 mm. The width from the center of the A1 well to the center of the H1 well can optionally range from 62 to 66 mm. The height of the receiver plate can optionally range from 19-23 mm. The height of the receiver plate (including the lip that inserts in the transfer adapter and/or in the standard 96 deep well plate) can optionally range from 20-24 mm. The rest of measurements follow the same sizes as the feeder plate.

The three pieces of the miniature system of the above examples can be made using 3D printing techniques for example, by using a 3D Systems Viper 2Si Stereolithography (SLA) machine (now ProJet 6000 HD, 3D Systems, Rock Hill, S.C.), using Accura ClearVue resin (Cat No. 24046-902, 3D Systems, Rock Hill, S.C.) in high resolution mode, Layer (Z) resolution 0.05 mm In plane (X-Y) 0.08 mm.

The system of the present disclosure allows for the identification of transcriptional difference within genotype; the identification of transcriptional differences among genotypes; the predication of gene interactions; the prediction of mode of action of compounds and the validation of hits of ultra HTS.

Those skilled in the art will recognize, or be able to ascertain using no more than routine experimentation, many equivalents to the specific embodiments of the invention described herein. Such equivalents are intended to be encompassed by the following claims.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

1. An apparatus for housing a small model organism in array format comprising:
an array of chambers joined by a solid support, wherein a bottom section of each chamber includes a round bottom well that has one or more holes extending therethrough that are: (a) of sufficiently large size to be permeable to liquid and (b) of sufficiently small size to prevent exit of the small model organism.

2. The apparatus of claim 1, further comprising a small model organism in at least one chamber of the array of chambers.

3. The apparatus as recited in claim 1, wherein the array comprises 96 chambers in an 8 row by 12 column format.

4. The apparatus as recited in claim 1, wherein the solid support is a plastic.

5. The apparatus as recited in claim 4, wherein the solid support is selected from the group consisting of a UV cure resin, a polystyrene or polypropylene and a coating of the substrate.

6. The apparatus of claim 2, wherein the small model organism is selected from the group consisting of Drosophila melanogaster, Daphnia, Hyalella, C. elegans and zebrafish.

7. The apparatus as recited in claim 1, wherein the solid support further comprises at least 3 alignment holes.

8. The apparatus as recited in claim 1, wherein the one or more holes formed in the round bottom are approximately 350 microns in size.

9. The apparatus as recited in claim 1, further comprising an adapter plate that allows for the interconnection of the array of chambers to a receiver plate, wherein the receiver plate comprises an array of circular deep wells.

10. The apparatus of claim 9, wherein the adapter plate comprises an array of square-to-round well adaptors.

11. The apparatus of claim 1, wherein a top of the array of chambers is covered by a removable layer that is impermeable to the small model organism.

12. A kit comprising a feeder plate for housing a small model organism in array format, a receiver plate, and instructions for its use, wherein the feeder plate comprises:
an array of chambers joined by a solid support, wherein the bottom of each chamber includes a round bottom well that has one or more holes that are: (a) of sufficiently large size to be permeable to liquid and (b) of sufficiently small size to prevent exit of the small model organism, and
the receiver plate comprises an array of circular deep wells and wherein the receiver plate interfaces with the feeder plate directly or via an adapter plate.

13. The kit of claim 12, wherein the array comprises 96 chambers in an 8 row by 12 column format.

14. The kit of claim 12, wherein the solid support is a plastic.

15. The kit as recited in claim 14, wherein the solid support is selected from the group consisting of a UV cure resin, a polystyrene or polypropylene and a coating of the substrate.

16. The kit as recited in claim 12, wherein the solid support for the array of chambers further comprises at least 3 alignment holes.

17. The kit as recited in claim 12, wherein the one or more holes of the round bottom are approximately 350 microns in size.

18. The kit as recited in claim 12, further comprising an adapter plate that allows for the interconnection of the array of chambers to the receiver plate, wherein the receiver plate comprises an array of circular deep wells.

19. A method for contacting a small model organism with a test compound, the method comprising introducing the test compound to a 96 well plate and contacting the 96 well plate including the test compound with an apparatus of claim 1, thereby contacting a small model organism with the test compound.
